(19) United States
(12) Patent Application Publication
(10) Pub. No.: US 2002/0037590 A1
(43) Pub. Date: Mar. 28, 2002

(54) Hygiene monitoring

(76) Inventors: Peter Leonard Grant, Needingworth (GB); Ramin Pirzad, St. Ives (GB)

Correspondence Address: SALIWANCHIK LLOYD & SALIWANCHIK, A PROFESSIONAL ASSOCIATION, 2421 N.W. 41ST STREET, SUITE A-1, GAINESVILLE, FL, 326066669

(21) Appl. No.: 09/882,626

(22) Filed: Jun. 15, 2001

Related U.S. Application Data
(63) Continuation-in-part of application No. PCT/GB99/04244, filed on Dec. 15, 1999.

(30) Foreign Application Priority Data
Jan. 11, 2001 (GB) 0100770.7

(51) Int. Cl. G01N 33/02

(52) U.S. Cl. 436/94; 436/95

(57) ABSTRACT

The subject invention provides for detecting the presence of food residue and/or microorganisms. The invention relates to hygiene monitoring and may be used to test a sample collected from an environment that originally was, or has subsequently been tested and been shown to be, free of sugar.

### REFERENCE TO RELATED APPLICATION

This Application is a continuation-in-part of PCT/GB99/04244, filed Dec. 15, 1999.

### FIELD OF THE INVENTION

This invention relates to hygiene monitoring, and in particular to an assay for the presence of food residues and/or microorganisms.

### BACKGROUND OF THE INVENTION

Hygiene monitoring procedures typically involves one of two procedures. One form of the first and oldest method comprises swabbing a surface, and analysing the sample taken up in the swab, for the presence of microorganisms. This method (conventional microbiology) takes 2-5 days to give a result and requires no instrumentation. The second and increasingly popular method again comprises swabbing a surface and analysing the sample taken up in the swab, for the presence of microorganisms and/or food residues. This method gives a result within a few minutes and requires instrumentation. The ability to detect the presence of food residues and/or microorganisms in the sample that has been taken up by swabbing relies on the presence of ATP. Reagents that are packaged within a device are brought in contact with the swabbed material and convert the ATP into light (bioluminescence) which is monitored by the instrument (luminometer). As disclosed in WO-A-94/25619, the amount of ATP may be amplified by reagents within the device, and the product converted to a detectable signal, a visible colour change. This colour change is mediated through the production of glucose. The preferred amplification reaction involves, inter alia, the amplification of ATP and then the conversion of glucose-6-phosphate to glucose, and the conversion of glucose, via a sequence of enzymatic reactions, to a coloured end point.

### SUMMARY OF THE INVENTION

It has now been found that, in certain circumstances, glucose itself or another sugar may be a sufficient indicator, for the purposes of an assay used in hygiene monitoring. According to the present invention, therefore, such an assay comprises the collection of a sample from a locus, and the determination of the presence of carbohydrate in the sample.

### DESCRIPTION OF THE INVENTION

In use of the invention, it may desirable to ensure that the sample is collected from an environment that originally was, or has subsequently been tested and shown to be, free of sugar. It may also be desirable that the locus to be tested is also assayed for the substantial absence of materials that may interfere in the novel assay, e.g. by inhibiting or by giving false positives, such as peroxide or reducing agents that may be incorporated in materials used to sanitise the locus.

The present invention can be practised utilising the same reagents as are disclosed in WO-A-94/25619, especially in so far as that relates to the conversion of glucose to a detectable signal. If desired, the reagents may exclude one or more of the components that are disclosed there for the conversion of ATP to glucose. A suitable device that can be used for the purposes of swabbing and detection is disclosed in WO-A-98/27196 and WO-A-99/31218.

The signal generated in the presence of glucose appears relatively rapidly, and can thus be distinguished, in addition to the advantage of providing a rapid response. The amplification of ATP needed to generate the signal is relatively slow.

Carbohydrates other than glucose alone may also be sufficient indicators of hygiene. Additionally, there are instances where certain carbohydrates are better indicators than glucose, in that levels of these sugars are higher than glucose in certain industry sectors. For example, in procedures involving milk processing, a major constituent is lactose. Residues of lactose that are not removed by cleaning regimes will provide a focus for microbial growth and potential contamination of product. Lactose can be converted to a visible signal through e.g. the conversion to glucose and galactose. Both galactose and glucose can be converted to a visible signal. In another example, “table sugar” (sucrose) may be added to products for taste. Again residues of sucrose not removed by cleaning regimes will provide a focus for microbial growth and potential contamination of product. Sucrose can be converted to glucose and fructose and the glucose is detected in the normal way. The invention can therefore product more specific and sensitive tests for hygiene than with glucose alone. In addition, this invention can be combined with reagents described in WO-A-94/25619 and above, to detect a range of carbohydrates, ATP and ADP, to give a more comprehensive test to the food industry.

The following Examples illustrate the utility of the present invention. All used the same amplification system, with a colour end-point, as disclosed in Example 1 of WO-A-94/25619.

### EXAMPLE 1

Swabs were taken from a variety of locations, and tested. In addition to determining the total viable count (TVC) and Enterobacteria (Enteros), contact plates were used alongside two devices. systemSURE (“sSURE”) is a portable hygiene monitoring system (available from Becton Dickinson) that uses bioluminescence as an endpoint. The “Pen Swab” is generally as disclosed in WO-A-99/31218. Results are shown in Table 1.

TABLE 1

| | | | sSURE | Pen Swab | |
|---|---|---|---|---|---|
| Location | TVC | Enteros | (RLU) | (min) | Agreement |
| Vegetable | 13 | 0 | 9462 (+) | + (Inst) | ✓ |
| chopping board | | | | | |
| Sweet preparation | Over | 75 | 625 (+) | + (Inst) | ✓ |
| area | | | | | |
| Dinner Plate | 0 | 0 | 158 (−) | − (14) | ✓ |
| Fridge door | Over | Over | 11353 (+) | + (Inst) | ✓ |
| handle | | | | | |
| Veg/General | 0 | 0 | 275 (−) | − (13) | ✓ |
| Storage | | | | | |
| Containers | | | | | |
| Uncooked meats | 21 | 0 | 219 (−) | + (1) | ✓ |
| chopping board | | | | | |
| Cooked meat | 12 | 0 | 4588 (+) | + (1) | ✓ |
| chopping board | | | | | |
| Handwash tap | ND | ND | 2451 (+) | + (1) | ✓ |
| (hot) | | | | | |

ND = no data
Inst = “instant” colour change
Threshold - 500 RLU

The data show that, in samples 1 and 4, the colour reagents produced an instant positive signal which, when compared to the bioluminescence results, is an indication of the present of glucose and not ATP. Sample 2 also gave a high total viable count (over enumeration limit) and 75 counts for Enteros, while giving 625 RLU for systemSURE and an immediate colour change for the pen. This again indicates the presence of glucose leading to contamination.

### EXAMPLE 2

The colour reagents were used to determine the level at which glucose was detectable. The reagents were activated in the presence of different concentrations of glucose. The absorbance values for a complete colour change were obtained within 120 seconds. Results are shown in Table 2.

TABLE 2

| Glucose (mM) | Glucose (μg) | Abs in Reader (OD) |
|---|---|---|
| 0.00 | 0.00 | 0.081 |
| 0.01 | 0.90 | 0.083 |
| 0.05 | 0.45 | 0.154 |
| 0.10 | 0.90 | 0.335 |
| 0.50 | 4.50 | 1.858 |
| 1.00 | 9.00 | 3.820 |
| 5.00 | 45.00 | 2.071 |
| 10.00 | 90.00 | 1.976 |
| 100.00 | 900.00 | 2.877 |

Swabs were taken from a variety of locations in a store, at different times of day and tested. Results are shown, for two different days, in Tables 3A and 3B. A large proportion of the data shows a rapid colour change (in seconds), characteristic of glucose detection. The corresponding RLU values do not show the presence of gross amounts of ATP, which is consistent with the detection of glucose.

TABLE 3A

| | 05.30 Pre-opening | | | | | 15.30 In-use | | | | | 22.30 Closedown - Pre-clean | | | | | Closedown - Post-clean | | | | |
|---|---|---|---|---|---|---|---|---|---|---|---|---|---|---|---|---|---|---|---|---|
| | | Swab | | | | | Swab | | | | | Swab | | | | | Swab | | | |
| | sSure | time | | Pass/Fail | | sSure | time | | Pass/Fail | | sSure | time | | Pass/Fail | | sSure | time | | Pass/Fail | |
| Location | RLU | to +ve | | ss | swab | RLU | to +ve | | ss | swab | RLU | to +ve | | ss | swab | RLU | to +ve | | ss | swab |
| Raw meat cutting | 10224 | >10 | min | F | P | 206743 | 5 | sec | F | F | | | | | | 44613 | 3 | min | F | F |
| board | | | | | | | | | | | | | | | | | | | | |
| Raw meat cutting | 4400 | >10 | min | F | P | 94339 | 5 | sec | F | F | | | | | | 19398 | 5 | min | F | F |
| board | | | | | | | | | | | | | | | | | | | | |
| Raw fish cutting | 760 | 9 | min | P | P | 916 | 10 | min | P | P | | | | | | 2888 | >10 | min | F | P |
| board | | | | | | | | | | | | | | | | | | | | |
| Raw fish cutting | 2368 | 9 | min | F | P | 2026 | 6 | min | F | P | | | | | | 4865 | >10 | min | F | P |
| board | | | | | | | | | | | | | | | | | | | | |
| Deli cooked food | 2724 | 15 | sec | F | F | 107 | 6 | min | P | P | 6991 | 15 | sec | F | F | 2699 | 2 | min | F | F |
| cutting board | | | | | | | | | | | | | | | | | | | | |
| Deli cooked food | 2395 | 15 | sec | F | F | 214 | 24 | sec | P | F | 2707 | 1 | min | F | F | 2403 | 1 | min | F | F |
| cutting board | | | | | | | | | | | | | | | | | | | | |
| Deli blue cheese | 2304 | 1 | min | F | F | 3525 | 10 | sec | F | F | 2200 | <5 | sec | F | F | 889 | 9 | min | P | P |
| cutting board | | | | | | | | | | | | | | | | | | | | |
| Deli blue cheese | 1686 | 1 | min | F | F | 693 | 10 | min | P | P | 2385 | <5 | sec | F | F | 589 | 9 | min | P | P |
| cutting board | | | | | | | | | | | | | | | | | | | | |
| Deli cooked | 134 | 7 | min | P | P | 1354 | 10 | sec | F | F | 42333 | <10 | sec | F | F | 65 | 6 | min | P | P |
| meat slicer | | | | | | | | | | | | | | | | | | | | |
| Deli cured | 591 | 9 | min | P | P | 1482 | 10 | sec | F | F | 258 | 20 | sec | P | F | 32 | 9 | min | P | P |
| meat slicer | | | | | | | | | | | | | | | | | | | | |
| Deli cheese | 353 | >10 | min | P | P | 2777 | 10 | sec | F | F | 1110 | 10 | sec | F | F | 1453 | 6 | min | F | P |
| cutter board | | | | | | | | | | | | | | | | | | | | |
| Deli cheese | 1099 | >10 | min | F | P | 2507 | 1 | min | F | F | 2548 | 3 | min | F | F | 1489 | 9 | min | F | P |
| cutter board | | | | | | | | | | | | | | | | | | | | |
| Cream room | 2110 | 30 | sec | F | F | 2229 | 5 | min | F | F | | | | | | 373 | 5 | sec | P | F |
| preparation table | | | | | | | | | | | | | | | | | | | | |
| Cream room | 1580 | 30 | sec | F | F | 2206 | 5 | min | F | F | | | | | | 801 | 10 sec | P | F | |
| preparation table | | | | | | | | | | | | | | | | | | | | |

1. A hygiene monitoring method, which comprises:
a) swabbing a surface with a swab;
b) collecting a sample taken up in the swab from the surface; and
c) determining the presence of a carbohydrate as a measure of food residues and/or microorganisms in the sample.

2. The method according to claim 1, wherein the carbohydrate is lactose or sucrose.

3. The method according to claim 1, wherein the carbohydrate is glucose.

4. The method according to claim 1, which comprises adding to the sample reagents that convert the carbohydrate to give a signal.

5. The method according to claim 4, wherein the reagents also convert ATP to give the signal and the signal is generated more quickly in the presence of the carbohydrate than in the presence of ATP.

6. The method according to claim 3, which further comprises the addition of reagents that convert ATP and carbohydrates to give a signal and wherein the signal is generated more quickly in the presence of the carbohydrate than in the presence of ATP.

7. The method according to claim 2, which further comprises the addition of reagents that convert ATP and carbohydrates to give a signal and wherein the signal is generated more quickly in the presence of the carbohydrate than in the presence of ATP.

8. The method according to claim 1, which further comprises the addition of reagents that convert ATP and carbohydrates to give a signal and wherein the signal is generated more quickly in the presence of the carbohydrate than in the presence of ATP.

9. The method according to claim 3, which comprises adding to the sample reagents that convert the carbohydrate to give a signal.

10. The method according to claim 2, which comprises adding to the sample reagents that convert the carbohydrate to give a signal.

11. The method according to claim 9, wherein the reagents also convert ATP to give the signal and the signal is generated more quickly in the presence of the carbohydrate than in the presence of ATP.

12. The method according to claim 10, wherein the reagents also convert ATP to give the signal and the signal is generated more quickly in the presence of the carbohydrate than in the presence of ATP.

13. The method according to claim 2, wherein the carbohydrate is lactose.

14. The method according to claim 2, wherein the carbohydrate is sucrose.

15. The method according to claim 4, wherein the carbohydrate is glucose.

16. The method according to claim 7, wherein the carbohydrate is lactose.

17. The method according to claim 7, wherein the carbohydrate is sucrose.
